(19) United States
(12) Patent Application Publication
(10) Pub. No.: US 2014/0336645 A1
(43) Pub. Date: Nov. 13, 2014

(54) FORCEPS WITH CONTINUOUS LATCH

(71) Applicant: GYRUS ACMI, INC., d/b/a Olympus Surgical Technologies America, Southborough, MA (US)

(72) Inventors: John Mensch, Plymouth, MN (US); Riyad Moe, Waunakee, WI (US); Jeffrey Nelson, Minneapolis, MN (US); Michael P. Baden, Long Lake, MN (US)

(21) Appl. No.: 14/270,538

(22) Filed: May 6, 2014

Related U.S. Application Data
(60) Provisional application No. 61/820,290, filed on May 7, 2013.

(51) Int. Cl. A61B 18/14

(52) U.S. Cl. CPC A61B 18/1445; A61B 2018/1452; A61B 2018/00607
USPC 606/52

(57) ABSTRACT

An article comprising: a housing comprising: i. a barrel portion, wherein a shaft capable of unidirectional or bidirectional movement is disposed within the barrel portion; wherein the shaft comprises a proximal end and a distal end; and wherein a pair of jaws is located at the distal end of the shaft; and ii. a plurality of controllers, wherein a jaw latch selector located under the barrel portion, connected to a shaft binder, is adapted for selecting between unidirectional and bidirectional movement of the shaft; and wherein a jaw controller located under the barrel portion is adapted to open and close the pair of jaws at the distal end of the shaft by movement of the shaft; wherein the shaft binder located in the barrel portion, disposed about the shaft, is connected to the jaw latch selector so that the shaft binder either binds upon the shaft to restrict movement of the shaft to unidirectional distal movement or does not bind upon the shaft so that the shaft moves bidirectionally; wherein the jaw controller and the jaw latch selector can be operated by fingers of the hand holding the housing; and wherein the shaft binder comprises a top portion and a bottom portion, wherein the top portion is held in a fixed position by the housing.

### FIELD

The present invention relates generally to surgical instruments, and more particularly to an ergonomic handle for an endoscopic or laparoscopic electrosurgical instrument housing a plurality of controllers.

### BACKGROUND

Different versions of minimally invasive electrosurgical instruments; i.e. forceps are known, for example U.S. Pat. No. 5,445,638, 5,735,849; 5,425,743; 5,499,998; 5,456,695; 5,417,203; 5,483,952; or 6,117,158, all of which are expressly incorporated by reference in their entirety herein for all purposes. When using an electrosurgical instrument, a surgeon may wish to adjust the amount of force by which he or she grasps tissue in the pair of jaws at the distal end of the instrument. To do so, the surgeon may want the jaw closing mechanism to have bidirectional movement; i.e. move forward and backward with his or her varying grip pressure. Alternatively, the surgeon may want the jaw closing mechanism to move unidirectionally, i.e. move forward with grip increased force, hold the grip when the surgeon removes his or her hand from the jaw closing mechanism, and continue the forward movement when the surgeon relieves the grip of the jaws.

Forceps with a mechanism switching from unidirectional movement to bidirectional are known in the art. For example, U.S. Pat. No. 5,456,695 teaches forceps with a locking plate that works as a unidirectional latching plate or U.S. Pat. No. 5,483,952 which teaches forceps with a trigger that temporarily overrides unidirectional mode. The problem is that such forceps do not have a set position for bidirectional mode; therefore, the device does not stay in bidirectional mode once the operator removes his or her hand from the trigger overriding the unidirectional mode. Furthermore, to operate such forceps, the operator must control numerous triggers. For example, a surgeon has to operate three triggers to move the shaft and open and close the pair of jaws at the end of the shaft when using the forceps as disclosed in the U.S. Pat. No. 5,735,849: the surgeon can actuate the buttons for opening and closing of the jaws with his or her index finger of the hand holding the forceps, but he or she has to use fingers of his or her other hand to actuate the trigger for operating the movement of the shaft.

Therefore, there is need for a surgical instrument which would allow for switching between unidirectional and bidirectional mode, overriding unidirectional mode, and holding the bidirectional mode by operating a single controller. Additionally, there is a need for an ergonomic housing which would allow operation of the forceps and all of its controllers with the fingers of the operator's hand holding the forceps.

### SUMMARY

One possible embodiment of the present teachings includes: an article comprising: a housing comprising: a barrel portion, wherein a shaft capable of unidirectional or bidirectional movement is disposed within the barrel portion; wherein the shaft comprises a proximal end and a distal end; and wherein a pair of jaws is located at the distal end of the shaft; and ii. a plurality of controllers, wherein a jaw latch selector located under the barrel portion, connected to a shaft binder, is adapted for selecting between unidirectional and bidirectional movement of the shaft; and wherein a jaw controller located under the barrel portion is adapted to open and close the pair of jaws at the distal end of the shaft by movement of the shaft; wherein the shaft binder located in the barrel portion, disposed about the shaft, is connected to the jaw latch selector so that the shaft binder either binds upon the shaft to restrict movement of the shaft to unidirectional movement or does not bind upon the shaft so that the shaft moves bidirectionally; wherein the jaw controller and the jaw latch selector can be operated by fingers of the hand holding the housing; and wherein the shaft binder comprises a top portion and a bottom portion, wherein the top portion is held in a fixed position by the housing.

Advantageous embodiments read from the dependent claims. Preferably, the top portion of the shaft binder is held in a slot within the housing. Preferably, the bottom portion of the shaft binder moves freely so that the jaw latch selector can switch from unidirectional movement to bidirectional movement of the shaft. Preferably, the shaft binder has a sharp inner edge so that it can bind the shaft. Preferably, the housing further comprises a detent part located in the barrel portion, wherein the jaw latch selector engages the detent part to slide into a detent pocket disposed on the jaw latch selector so that the detent part holds the jaw latch selector in the bidirectional movement of the shaft. Preferably, the detent part is a spring-loaded detent pin. Preferably, the jaw latch selector has a first position engaging the shaft binder to bind the shaft so that the shaft travels unidirectionally only. Preferably, the jaw latch selector has a second position not engaging the shaft binder to bind the shaft so that the shaft travels bidirectionally. Preferably, the jaw latch selector is located under the barrel portion in such a way that it can be reached by the index finger of the hand holding the housing. Preferably, the jaw latch selector is shaped as a lever so that it can be pushed by an index finger towards the bottom of the barrel portion. Preferably, the housing further comprises a rotational controller for rotating the pair of jaws. Preferably, the rotational controller is located in the housing in such a way that it can be reached by an index finger of the hand holding the housing. Preferably, the housing further comprises a cut trigger located under the barrel portion, wherein the cut trigger is adapted to control a cutting element attached to the distal end of the shaft. Preferably, the jaw latch selector, the jaw controller, a rotational controller, a cut trigger, and a coagulation controller are independent of each other, can be operated by the fingers of the hand holding the housing, or both. Preferably, the housing further comprises a stationary handle extending from the barrel portion.

The present teachings provide a surgical instrument that is substantially ergonomic so that all of the controllers may be operated with fingers of the hand holding the instrument; therefore occupying only one hand of the operator. The present teachings provide a surgical instrument with a controller which allows for switching between unidirectional mode and bidirectional mode. In addition, the same controller allows for the bidirectional mode to be biased even when the operator releases the controller. Another advantage of the present invention is that the invention allows the operator to move the shaft unidirectionally or bidirectionally and to open and close the pair of jaws located at the distal end of the shaft with just two controllers.

### BRIEF DESCRIPTION OF THE DRAWINGS

FIG. 1 illustrates one example of an article including a housing of the teachings herein.

FIG. 2 illustrates one example of a set of instruments located at the distal end of the shaft.

FIGS. 3A and 3B illustrate examples of a shaft binder of the teachings herein.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present teachings provide a surgical or an electrosurgical instrument, such as a laparoscopic or endoscopic surgical forceps, comprising of a housing containing a barrel portion and a plurality of controllers. The housing may have any size, shape, configuration, or a combination thereof so that the housing can be gripped with a left hand, right hand, or either. Preferably, the housing has an ergonomic shape so that the housing accommodates all fingers of a right hand or left hand. The housing may be made of any material commonly utilized in housings of forceps, such as plastic, metal, or the like. Preferably, the housing is made of a lightweight material which can withstand a variety of sterilization techniques. Preferably, the housing is made of an engineering plastic, more preferably acrylonitrile butadiene styrene (ABS), polycarbonate (PC), or a blend of ABS and PC.

The barrel portion of the housing accommodates working components used to operate the invention. For example, the barrel portion may accommodate a shaft, a plurality of controllers, various parts of the controllers' mechanism, the like, or a combination thereof. The barrel portion can be anything that performs this function. The barrel portion forms the top portion of the housing. The barrel portion may extend into a stationary handle in such a way that when held by a left hand, right hand, or either, the barrel portion is situated above the index finger, and the thumb's thenar presses against the stationary handle.

The shaft accommodates a variety of hand-held surgical instruments, rotates, moves unidirectionally and bidirectionally. The shaft may be anything that performs this function. Preferably, the shaft is an elongated distally moving tube which extends from the barrel portion of the housing. The shaft has a proximal end and a distal end. The proximal end is a part located close to the housing, within the housing, or both. The distal end is a part located furthest from the housing. The proximal end of the shaft may be disposed within the barrel portion of the housing. At the distal end, the shaft may accommodate a variety of elements and hand-held instruments, such as a pair of jaws, a cutting instrument, surgical scissors, a laparoscopic entry device, unarticulating probes, an electrosurgical needle, the like, or a combination thereof. The shaft may further accommodate a conductor of electricity such as a wire extending from the proximal end to the instruments located at the distal end. The shaft may be made of any material commonly utilized in shafts, such as metal, plastic or the like. Preferably, the shaft is made of stainless steel. The shaft may have a coating, for example a polytetrafluoroethylene (PTFE) coating. The shaft may be supported by a shaft supporting bushing which may hold the shaft in place within the barrel portion of the housing.

The pair of jaws is an element capable of opening, closing, rotating, clamping tissue, or a combination thereof. The pair of jaws can be anything that performs this function. The pair of jaws may comprise a first jaw and a second jaw located at the distal end of the shaft. The pair of jaws may include a pair of electrodes disposed on the first jaw and the second jaw. The jaws may be opened, closed, rotated, or a combination thereof by operation of one or more controllers disposed within the housing. For example, a jaw controller may control the clamping function of the pair of jaws located at the distal end of the shaft.

The plurality of controllers serves to operate various instruments located at the distal end of the shaft. For example, opening and closing the pair of jaws, rotating the pair of jaws, clamping tissue, cutting, coagulating, entering tissue or an opening within tissue, injecting, sucking, a combination thereof, or the like. The plurality of controllers can be anything that performs this function. The plurality of controllers may comprise a jaw latch selector, a jaw controller, a rotational controller, a cut trigger, a coagulation controller, a combination thereof, or the like. The plurality of controllers may be independent of each other. The plurality of controllers is disposed within the barrel portion, the housing, or both. The plurality of controllers may be disposed within, on, in, or a combination thereof of the barrel portion. Preferably, the plurality of controllers is placed in such a way that the housing with the plurality of controllers has an ergonomic shape. More preferably, the plurality of controllers is placed in such a way that all the controllers can be operated by the same hand holding the housing. The plurality of controllers may be connected to the controllers' mechanisms, for example a latching mechanism, a cutting mechanism, a clamping mechanism, or the like.

A jaw latch selector allows the operator to select a unidirectional or bidirectional movement of the shaft, override unidirectional mode when selected, and hold bidirectional mode. The jaw latch selector can be anything that performs this function. The jaw latch selector is a controller located within the housing which is a part of the latching mechanism. The jaw latch selector is located under the barrel portion. The jaw latch selector may be attached to the barrel portion with any method of attachment which renders the jaw latch selector capable of performing its function, for example a fastener, a pin, the like, or a combination thereof. The jaw latch selector may have any size, shape, configuration, or a combination thereof so that it can be operated by a finger of the operator's hand holding the housing. Preferably, the jaw latch selector is secured to pivot in the housing. Preferably, the jaw latch selector is shaped like a lever to be operated by an index finger of the hand holding the housing. To choose a bidirectional mode, the operator's index finger pushes the jaw latch selector against the bottom of the barrel portion. To choose a unidirectional mode, the operator does not push the jaw latch selector against the barrel portion. The operator may switch from the bidirectional mode to the unidirectional mode by removing the index finger from the pushed jaw latch selector. To hold the bidirectional mode, the operator's index finger pushes the jaw latch selector towards the barrel portion so far that the jaw latch selector engages a detent part which holds the jaw latch selector in the bidirectional position. The jaw latch selector may be connected to a variety of components within the housing. Preferably, the jaw latch selector is connected to other components of the latching mechanism. More preferably, the jaw latch selector is connected to a detent part, a shaft binder, and a shaft binder reset. The jaw latch selector may comprise a detent pocket.

A detent pocket engages the detent part and biases the jaw latch selector in bidirectional mode. The detent pocket can be anything that performs this function. The detent pocket may be a small depression located on the jaw latch selector facing the detent part. Preferably, the detent pocket is located on the jaw latch selector so that the detent pocket engages the detent part and biases the jaw latch selector in bidirectional mode.

A detent part biases the jaw latch selector which holds the jaw latch selector in bidirectional mode. The detent part may be anything that performs this function. A detent part may be a small elongated part of the latching mechanism disposed within the barrel, connected to the jaw latch selector. When the operator pushes the jaw latch selector towards the bottom of the barrel portion so far that the detent pocket engages the detent part, the detent part holds the jaw latch selector in bidirectional mode. The detent part may have any size, shape, configuration, or a combination thereof so that it can be engaged by the detent pocket located on the jaw latch selector. Preferably, the detent part is a detent pin, a spring-loaded pin, a separate spring, an integral plastic lead spring, the like, or a combination thereof.

A shaft binder serves to bind the shaft which limits the shaft's travel to unidirectional only. The shaft binder can be anything that performs this function. For example, a shaft binder may be a lock washer or a plate. The shaft binder is disposed within the barrel portion, surrounding the shaft, being held in a fixed position by a housing, by a slot that restrains the shaft binder's movement on either or both sides, by a pivoting pin restricting shaft binder's movement on a pivoting axel, by a shaft binder reset such as a biasing spring, the like, or a combination thereof. The shaft binder is a part of the latching mechanism. The shaft binder may comprise a top portion and a bottom portion. Preferably, the shaft binder is biased towards one direction by a shaft binder reset. When the jaw latch selector is not pushed, the shaft binder may be biased into one direction by a shaft binder reset preventing movement of the shaft binder so that the shaft binder binds upon the shaft and limits the shaft's travel to unidirectional only. In the fixed position, the shaft binder is in a clutch orientation and the top portion is held in a fixed position, for example within a slot within the housing. When the jaw latch selector is pushed, the compressible energy provided by the shaft binder reset to hold the shaft binder in place is overcome, the shaft binder is in the non-clutch orientation, does not bind upon the shaft, and allows bidirectional movement of the shaft. By pushing and releasing the jaw latch selector, the operator causes the bottom portion of the shaft binder to move back and forth, switching between the clutch orientation and the non-clutch orientation and switching between the unidirectional and bidirectional movement of the shaft. The shaft binder, the shaft binder's top portion and/or bottom portion may have any shape, size, configuration, or a combination thereof so that the shaft binder can bind upon the shaft. For example, the shaft binder may be round, square, rectangular, regular, or irregular, symmetrical, or asymmetrical, with at least one opening to surround and engage the shaft. The opening within the shaft binder may have any shape, size, configuration, or a combination thereof so that the opening surrounds, engages, and binds upon the shaft. Preferably, the shaft is round and the opening is circular. Alternatively, the shaft binder may have a rectangular shape with an irregularly shaped opening. In another embodiment, the shaft binder's top portion may be straight, and the bottom portion may be rounded. The shaft binder may be made of any material which will provide the shaft binder with a sharp inner edge so that the shaft binder may bind the shaft. Preferably, the shaft binder is made of stainless steel.

A shaft binder reset provides compressible energy to hold the shaft binder in place to prevent movement of the shaft binder when the shaft moves by biasing the shaft binder into one direction. The shaft binder reset returns the shaft binder from the non-clutch orientation into the clutch orientation. The shaft binder reset can be anything that performs these functions. The shaft binder reset may be any size, shape, configuration, or a combination thereof so that the shaft binder reset may be placed around the shaft within the housing, more preferably between a shaft support bushing and the shaft binder. For example, the shaft binder reset may be a flexible compressive foam pad. Preferably, the shaft binder reset is a biasing spring.

The housing comprises a jaw controller. A jaw controller controls the clamping function of the pair of jaws located at the distal end of the shaft. The jaw controller actuates movement of the shaft back and forth which closes and opens the pair of jaws. The jaw controller can be anything that performs these functions. The jaw controller is a controller disposed below the barrel portion pivoting with respect to the stationary handle. The jaw controller may be any size, shape, configuration, or a combination thereof. Preferably, the jaw controller is a lever. More preferably, the jaw controller comprises an ergonomic finger loop so that the finger loop accommodates one or more fingers of the operator's hand holding the housing. Preferably, the operator's middle finger, ring finger, and small finger are engaged in the finger loop while the operator uses the housing. Preferably, the jaw controller pivots with respect to the housing. Alternatively, the jaw controller may move by means of four-bar mechanism or translating mechanism. When the operator depresses the jaw controller, the jaw controller pivots rearward with respect to the housing, towards the stationary handle, and the pair of jaws closes. The operator may reopen the pair of jaws by releasing the jaw controller so that the jaw controller pivots forward, away from the stationary handle. The jaw controller may be made of any material commonly utilized in jaw controllers of forceps, such as plastic. The finger loop may be made of any material which provides adequate support and grip to the operator's fingers without causing discomfort upon applying pressure when depressing the lever, for example rubber. The jaw controller is part of a clamping mechanism which may further comprise parts enabling transfer of the jaw closing force from the jaw controller to the pair of jaws, such as a drive link, a slide block, and an inside barrel member. The clamping mechanism may further comprise a jaw controller reset.

A drive link enables transfer of the jaw closing force from the jaw controller to the pair of jaws. The drive link may transfer the jaw closing force from the jaw controller to the slide block. The drive link can be anything that performs this function. Preferably, the drive link is an elongated arcuate part disposed within the barrel portion connecting the jaw controller with the slide block. A slide block enables transfer of the jaw closing force from the jaw controller to the pair of jaws. The slide block may transfer the jaw closing force from the drive link to the shaft. The slide block can be anything that performs this function. Preferably, the slide block is a circular member disposed around the shaft within the barrel portion connecting the drive link with the shaft. An inside barrel member enables transfer of the jaw closing force from the jaw controller to the pair of jaws. The inside barrel member is secured to the shaft, for example, the inside barrel member may be molded to the shaft. Preferably, the inside barrel member and the slide block are free to rotate around each other while not being secured together. Even more preferably, the inside barrel member rotates inside of the slide block and the slide block driven by the drive link moves the inside barrel member axially. A jaw controller reset returns the pair of jaws into the pair of jaws' original position. The jaw controller reset can be anything performing this function. Preferably, the jaw controller reset is a biasing spring.

The housing may further comprise a rotational controller. The rotational controller rotates the pair of jaws at the distal end of the shaft. The rotational controller can be anything that performs this function. The rotational controller may be of any size, shape, configuration, or a combination thereof. Preferably, the rotational controller is a circular knob disposed within the housing, protruding from the housing on the left side, right side, or both. The rotational controller may be actuated by operator's one or more fingers of the left hand, right hand, or either. Preferably, the rotational controller is actuated by the index finger of the operator's hand holding the housing. Preferably, the rotational controller is secured to the inside barrel member so that when the operator actuates the rotational controller, the inside barrel member secured to the shaft with the pair of jaws at the distal end rotates. The rotational controller may be made of any material commonly utilized in forceps' rotational controllers, such as plastic, metal, rubber, or the like. The surface of the rotational controller may be adjusted to allow for a better grip, for example, the surface may be provided with ridges.

The housing may further comprise a cut trigger. A cut trigger actuates cutting function of a cutting element which may be located at the distal end of the shaft. The cut trigger can be anything that performs this function. The cut trigger may be a controller disposed below the barrel portion. A cut trigger may be located where the barrel portion extends to the stationary handle and may be attached to the housing with any fastener, for example with one or more pins. The cut trigger may be any size, shape, configuration, or a combination thereof. Preferably, the cut trigger has ergonomic shape, for example, the cut trigger may comprise a curved portion to ergonomically accommodate the operator's index finger. When the operator depresses the cut trigger, a cutting element is actuated at the distal end of the shaft. The cut trigger is a part of a cutting mechanism which enables transfer of cutting force from the cut trigger to the cutting element, such as a cut blade rod, a cutter pivot, and a connector member. The cutting element cuts the tissue. The cutting element can be anything that performs this function. For example, the cutting element may be a blade. The cutting element may be located at the distal end of shaft, for example within or behind the pair of jaws. The connector member enables transfer of cutting force from the cut trigger to the cutting element. The connector member may transfer the cutting force from the cut trigger to the cutter pivot. The connector member can be anything that performs this function. Preferably, the connector member is an elongated part disposed within the housing, connecting the cut trigger with a cutter pivot. The cutter pivot enables transfer of cutting force from the cut trigger to the cutting element. The cutter pivot may transfer the cutting force from the connector member to the cut blade rod. The cutter pivot can be anything that performs this function. Preferably, the cutter pivot is an element disposed within the housing, connecting the connector member with the cut blade rod. The cut blade rod enables transfer of cutting force from the cut trigger to the cutting element. The cut blade rod may transfer the cutting force from the cutter pivot to the cutting element. The cut blade rod can be anything that performs this function. Preferably, the cut blade rod may be a rod disposed within the barrel portion.

The housing may further comprise a coagulation controller. A coagulation controller operates a conductor of electricity adapted for conducting electricity to an instrument attached at the distal end of the shaft. A coagulation controller can be anything that performs this function. Preferably, a coagulation controller is a button disposed on the barrel portion, on a stationary handle, or both. Preferably, the coagulation controller is located on top of the barrel portion in such a way that it can be reached by a thumb or index finger of the operator's hand holding the housing. The housing may further comprise a power cable strain relief which prevents damage to a power cable. The power cable strain relief can be anything that performs this function. Preferably, the power cable strain relief is a mechanical anchor.

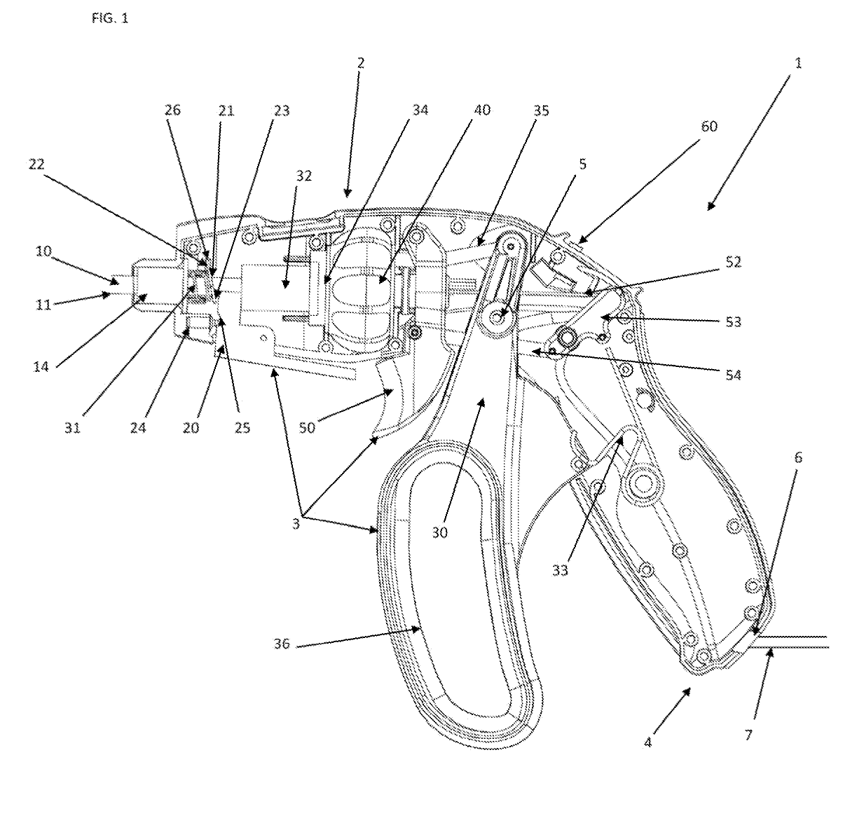

FIG. 1 illustrates one example of an article including a housing (1) of the teachings herein. As illustrated, the housing (1) comprises a barrel portion (2) extending to a stationary handle (4), and a plurality of controllers (3). The barrel portion (2) accommodates various parts of the housing (1) such as a shaft (10) comprising a proximal end (11). The shaft is supported by a shaft support bushing (14) located within the barrel portion (2). The operator may select a unidirectional or bidirectional movement of the shaft (10) by operating a jaw latch selector (20) located below the barrel portion (2). The jaw latch selector (20) is connected to other components of the latching mechanism: the shaft binder (21) surrounding the shaft (10) within the barrel portion (2), and the detent part (24) located within the barrel portion (2). The shaft binder (21) is biased into one direction by a shaft binder reset (31) which is a biasing spring surrounding the shaft (10) located between the shaft support bushing (14) and the shaft binder (21). The shaft binder (21) comprises a top portion (22) which is held in a fixed position by a slot (26) within the housing (1). The shaft binder (21) is binding upon the shaft (10) and limiting movement of the shaft (10) to unidirectional only when the jaw latch selector (20) is not pushed. The shaft binder (21) further comprises a bottom portion (23) which pivots freely when the jaw latch selector (21) is pushed so that the shaft binder (21) does not bind upon the shaft (10) and allows bidirectional movement of the shaft (10). The jaw latch selector (20) further comprises a detent pocket (25) which engages the detent part (24) biasing the jaw latch selector in bidirectional mode. The jaw latch selector (20) is to be actuated by operator's index finger. The housing (1) further comprises a jaw controller (30) located below the barrel portion (2), which is capable of actuating movement of the shaft (10) and closing and opening a pair of jaws (not depicted) located at the distal end of the shaft (10). The jaw controller (30) comprises a finger loop (36) accommodating the middle finger, the ring finger, and the small finger of the operator's hand holding the housing (1). The jaw controller (30) is connected to other parts of the jaw closing mechanism: the drive link (35), the slide block (34), the inside barrel member (32), all of which are disposed within the barrel member (2), and the jaw controller reset (33) disposed within the stationary handle (4). The housing (1) further comprises a rotational controller (40) located in the central portion of the barrel portion (2). The rotational controller (40) is to be actuated by operator's index finger. The rotational controller (40) is secured to the inside barrel member (32) which is secured to the shaft (10). The rotational controller (40) rotates the pair of jaws (not depicted) located at the distal end of the shaft (10). The housing (1) further comprises a cut trigger (50) located under the barrel portion (2) to be actuated by operator's index finger. The cut trigger (50) is connected to a connector member (54), a cutter pivot (53), and a cut blade rod (52) which are all disposed within the barrel portion (2) and allow for cutting function of a cutting element located at the distal end of the shaft (10). The housing further comprises a coagulation controller (60) located at the top of the barrel portion (2). In addition, the housing (1) comprises a power cable strain relief which serves as a mechanical anchor (6) preventing damage to power cables (7). The plurality of controllers, such as the jaw latch selector (20) and the jaw controller (30) are attached to the housing with pins (5).

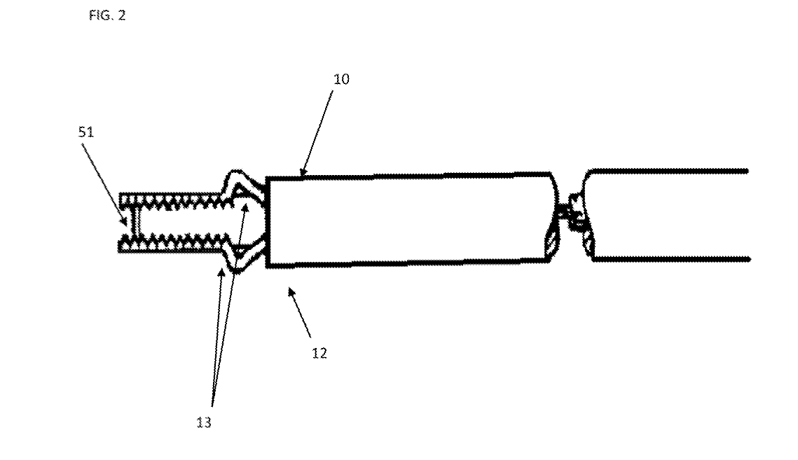

FIG. 2 illustrates one example of a set of instruments located at the distal end of shaft. As illustrated, the shaft (10) has a pair of jaws (13) and a cutting element (51) located at the distal end of the shaft (12).

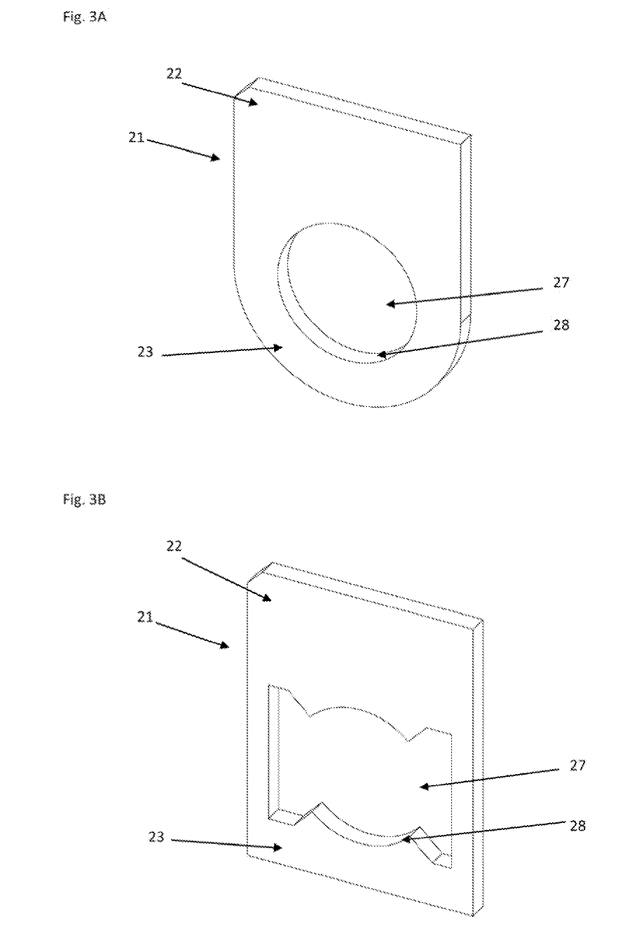

FIGS. 3A and 3B illustrate examples of a shaft binder of the teachings herein. As illustrated in FIG. 3A, a shaft binder's top portion (22) may be straight, the bottom portion (23) may be rounded, and the shaft binder (21) may have a circular opening (27) to surround and bind upon the shaft (not depicted). As illustrated in FIG. 3B, the shaft binder (21) may have a rectangular shape with straight top portion (22), straight bottom portion (23), and the opening (27) may have an irregular shape. The shaft binder (21) has a sharp inner edge (28) so that the shaft binder (21) can bind the shaft (10).

To operate the invention, the surgeon can activate different mechanisms independent of each other, for example a latching mechanism, a clamping mechanism, or a cutting mechanism. In addition, the surgeon may activate additional mechanisms depending on what type of a surgical instrument is accommodated at the distal end of the shaft. To choose a unidirectional or bidirectional mode, the surgeon presses or releases the jaw latch selector located under the barrel portion of the housing, reachable by the surgeon's index finger of the hand holding the housing. When the surgeon does not press on the jaw latch selector, a shaft binder is in the clutch orientation. In the clutch orientation, the shaft binder may be biased in one direction by a shaft binder reset such as a biasing spring and/or the shaft binder's top portion may be held in a fixed position, for example by a slot within the housing. The shaft binder's inner sharp edge binds upon the shaft and limits the shaft's travel to a forward motion. To switch from unidirectional to bidirectional mode, the surgeon presses the jaw latch selector towards the barrel portion, pushes the shaft binder into a non-clutch position so that the shaft binder does not bind upon the shaft anymore, and the shaft can move forward and backward. The surgeon can switch back to unidirectional mode by releasing the jaw latch selector. The shaft binder reset then returns the shaft binder into its original position by biasing the shaft binder in one direction. Alternatively, the surgeon can push the jaw latch selector so far towards the barrel portion that the jaw latch selector's detent pocket engages a detent part located within the housing. The detent part holds the jaw latch selector in the bidirectional mode, allowing the shaft to move forward and backward.

The surgeon may rotate the pair of jaws at the end of the shaft by operating the rotational controller. The surgeon places his or her index finger on the rotational controller which is disposed within the housing, protruding on the left side, right side, or both from the housing. By moving his or her index finger on the sides of the rotational controller, the surgeon rotates the rotational controller.

The surgeon may operate the movement of the shaft, advance the shaft distally or retreat the shaft towards the surgeon, and open and close the pair of jaws by operating the jaw controller. Movement of the shaft opens and closes the pair of jaws located at the distal end of the shaft. The jaw controller is located under the barrel portion, and preferably accommodates the middle finger, the ring finger, and the small finger of the surgeon's hand holding the housing. By pressing the jaw controller, the surgeon moves the jaw controller towards the stationary handle. This in turn activates other parts of the clamping mechanism, such as a drive link, a slide block, and an inside barrel member, and transfers jaw closing force from the jaw controller to the shaft with the pair of jaws at the shaft's distal end. To open the pair of jaws, the surgeon releases pressure he or she applied to the jaw controller which activates a jaw controller reset, such as a biasing spring, which moves the jaw controller forward, releases other parts of the jaw clamping mechanism and opens the pair of jaws. The jaw latch selector and the jaw controller may be operated at the same time; therefore allowing the surgeon to move forward and backward with increased or decreased pressure on the tissue.

The surgeon may operate the cutting mechanism by operating the cut trigger located under the barrel portion. The surgeon pushes the cut trigger by his or her index finger of the hand holding the housing. This in turn activates other parts of the cutting mechanism, such as a connector member, a cutter pivot, and a cut blade rod, and transfers the cutting force from the cut trigger to a cutting element at the distal end of the shaft. The surgeon would not operate the cut trigger and the jaw latch selector at the same time.

1. An article comprising:
a housing comprising:
i. a barrel portion,
wherein a shaft capable of unidirectional or bidirectional movement is disposed within the barrel portion,
wherein the shaft comprises a proximal end and a distal end, and wherein a pair of jaws is located at the distal end of the shaft, and
ii. a plurality of controllers,
wherein a jaw latch selector located under the barrel portion, connected to a shaft binder, is adapted for selecting between unidirectional and bidirectional movement of the shaft, and
wherein a jaw controller located under the barrel portion is adapted to open and close the pair of jaws at the distal end of the shaft by movement of the shaft,
wherein the shaft binder located in the barrel portion, disposed about the shaft, is connected to the jaw latch selector so that the shaft binder either binds upon the shaft to restrict movement of the shaft to unidirectional movement or does not bind upon the shaft so that the shaft moves bidirectionally;
wherein the jaw latch selector and the jaw controller can be operated by fingers of the hand holding the housing; and
wherein the shaft binder comprises a top portion and a bottom portion, wherein the top portion is held in a fixed position by the housing.

2. An article according claim 1, wherein the top portion of the shaft binder is held in a slot within the housing.

3. An article according to claim 2, wherein the bottom portion of the shaft binder moves freely so that the jaw latch selector can switch from unidirectional movement to bidirectional movement of the shaft.

4. An article according to claim 1, wherein the shaft binder has a sharp inner edge so that it can bind the shaft.

5. An article according to claim 1, wherein the housing further comprises a detent part located in the barrel portion, wherein the jaw latch selector engages the detent part to slide into a detent pocket disposed on the jaw latch selector so that the detent part holds the jaw latch selector in the bidirectional movement of the shaft.

6. An article according to claim 5, wherein the detent part is a spring-loaded detent pin.

7. An article according to claim 1, wherein the jaw latch selector has a first position engaging the shaft binder to bind the shaft so that the shaft travels unidirectionally only.

8. An article according to claim 1, wherein the jaw latch selector has a second position not engaging the shaft binder to bind the shaft so that the shaft travels bidirectionally.

9. An article according to claim 1, wherein the jaw latch selector is located under the barrel portion in such a way that it can be reached by the index finger of the hand holding the housing.

10. An article according to claim 1, wherein the jaw latch selector is shaped as a lever so that it can be pushed by an index finger towards the bottom of the barrel portion.

11. An article according to claim 1, wherein the housing further comprises a rotational controller for rotating the pair of jaws.

12. An article according to claim 11, wherein the rotational controller is located in the housing in such a way that it can be reached by an index finger of the hand holding the housing.

13. An article according to claim 1, wherein the housing further comprises a cut trigger located under the barrel portion, wherein the cut trigger is adapted to control a cutting element attached to the distal end of the shaft.

14. An article according to claim 1, wherein the jaw latch selector, the jaw controller, a rotational controller, a cut trigger, and a coagulation controller are independent of each other, can be operated by the fingers of the hand holding the housing, or both.

15. An article according to claim 1, wherein the housing further comprises a stationary handle extending from the barrel portion.
